(19) United States
(12) Patent Application Publication
(10) Pub. No.: US 2013/0006180 A1
(43) Pub. Date: Jan. 3, 2013

(54) Nanoporous Membrane Responsive to Electrical Stimulation and Method for Manufacturing the Same

(75) Inventors: Gumhye Jeon, Jinhae-si (KR); Jin Kon Kim, Gyeongju-si (KR); Seung Yun Yang, Daegu (KR); Jinseok Byun, Daejeon (KR)

(73) Assignee: POSTECH ACADEMY-INDUSTRY FOUNDATION, Pohang-city (KR)

(21) Appl. No.: 13/247,615

(22) Filed: Sep. 28, 2011

(30) Foreign Application Priority Data
Jun. 29, 2011 (KR) 10-2011-0063990

(51) Int. Cl. A61M 5/00; B05D 3/06

(52) U.S. Cl. 604/131; 427/2.21

(57) ABSTRACT

The present invention relates to a nanoporous membrane for flux control in response to electrical stimulation. The nanoporous membrane includes a supporting layer with a plurality of pores; and an electrically responsive layer that is connected to around the entrances of the pores and undergoes a volume change by oxidation or reduction caused by electrical stimulation to thereby lead to a change in pore size.

### CROSS-REFERENCE TO RELATED APPLICATION

This application claims priority to and the benefit of Korean Patent Application No. 10-2011-0063990 filed in the Korean Intellectual Property Office on Jun. 29, 2011, the entire contents of which are incorporated herein by reference.

### BACKGROUND OF THE INVENTION

(a) Field of the Invention

The present invention relates to a nanoporous membrane for flux control in response to electrical stimulation and a method for manufacturing the same.

(b) Description of the Related Art

In general, drug administration methods include oral administration, parenteral administration, local application, etc. In oral and parenteral (e.g., injection) administration, drug concentration distribution of the body shows a high concentration at an early stage and a low concentration at a later stage. Accordingly, high concentrations may lead to side effects caused by excessive administration, and low concentrations may lead to drug waste if they reach below the effective therapeutic dose.

Moreover, drug administration methods include sustained drug delivery and pulsatile drug delivery depending on the manner of administration. The purpose of sustained drug administration is to release drugs for a long time as constant concentration, and the purpose of pulsatile drug delivery is to release drugs periodically or discontinuously depending on the point of time of drug administration.

In field of pulsatile drug delivery, the discontinuous drug administration requires material whose phase is changeable in response to stimulation. Applicable stimulation includes temperature, pH, degradation rate, bio-material, light, sound, magnetism, electrical stimulation, and so on. However, such as temperature, pH, degradation rate, bio-material cannot be controlled artificially in vivo. Therefore, it is desirable to use sound, light, magnetism, and electrical stimulation to freely control in vivo stimulation. Among them, electrical stimulation has the advantage of portability over other stimulus because expensive and special device are not required to apply stimulation.

Devices for releasing drugs responding to electrical stimulation that have been studied so far include a method for releasing a drug by loading drug as layer-by-layer manner and applying electrical stimulation, a method for releasing drugs by loading the drug on degradable polymer through electrospinning, enclosing the drug in conducting polymer and released by applying electrical stimulation, a method for releasing a drug by loading the drug on gel degradable upon electrical stimulation, and controlling degradation rate, and a method for releasing a drug by forming a micro-sized drug reservoir by a complicated lithography process and applying electrical stimulation at a desired point of time to remove a metal cap covering the reservoir.

However, the conventional devices for releasing drugs responsive to electrical stimulus have the disadvantages of time-consuming and expensive fabrication method, a limited dose of drug that can be loaded, incapability of controlling a precise dose, and a limited number of times of opening and closing.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide a nanoporous membrane including pores, which is capable of freely controlling the size of the pores by electrical stimulation and enables stable and discontinuous release of a drug by flow control, and a method for manufacturing the nanoporous membrane.

An exemplary embodiment of the present invention provides a nanoporous membrane including: a supporting layer with a plurality of pores; and an electrically responsive layer that is connected to around the entrances of the pores and undergoes a volume change by oxidation or reduction caused by electrical stimulation to thereby lead to a change in pore size.

The supporting layer may be made of anodic aluminum oxide membrane, and the electrically responsive layer may include an electrode layer connected to around the entrances of the pores and a conducting polymer layer that is connected to the electrode layer and undergoes a volume change by oxidation or reduction due to electricity applied to the electrode layer.

The electrode layer may include gold, and gold may be formed around the entrances of the pores by either thermal deposition or sputtering.

The conductive polymer layer may include a conducting polymer and a dopant.

The conducting polymer may include polypyrrole, and the dopant may include dodecylbenzenesulfonate anions.

The nanoporous membrane may further include an impact absorbing layer connected to the supporting layer.

The impact absorbing layer may include polymer.

The electrically responsive layer may contract in volume if oxidized by electrical stimulation.

The electrically responsive layer may expand in volume if reduced by electrical stimulation.

Another exemplary embodiment of the present invention provides a method for forming a nanoporous membrane, the method including: forming a supporting layer with a plurality of pores; and forming an electrically responsive layer that is connected to around the entrances of the pores and oxidized or reduced by electrical stimulation.

The forming of a supporting layer may include forming pores using an anodic aluminum oxide membrane.

The forming of an electrically responsive layer may include: forming an electrode layer connected to around the entrances of the pores; and forming a conducting polymer layer connected to the electrode layer.

The electrode layer may include gold, and gold may be formed around the entrances of the pores by either thermal deposition or sputtering.

The forming of an electrically responsive layer may include electrically polymerizing the oxidized conducting polymer with the dopant.

The conducting polymer may include polypyrrole, and the dopant may include dodecylbenzenesulfonate anions.

The method may further include connecting an impact absorbing layer to the supporting layer.

The nanoporous membrane according to an exemplary embodiment of the present invention can precisely control the amount of drug release because the pore size can be freely adjusted by oxidation and reduction that occurs reversibly by electrical stimulation.

Moreover, the method for forming the nanoporous membrane according to another exemplary embodiment of the present invention enables it to relatively freely control the size of the pores and the thickness of the nanoporous membrane, thus simplifying the manufacture of the nanoporous membrane.

### BRIEF DESCRIPTION OF THE DRAWINGS

FIG. 1A is a perspective view schematically showing a supporting layer according to the present invention.

FIG. 1B is a perspective view schematically showing connection of an electrode layer to the supporting layer of FIG. 1A.

FIG. 1C is a perspective view schematically showing connection of a conducting polymer layer to the electrode layer of FIG. 1B.

FIG. 2A is a field emission-scanning electron microscopic image of the plane and cross-sectional view of the supporting layer of FIG. 1A.

FIG. 2B is a field emission-scanning electron microscopic image of the plane and cross-sectional view of the connection of the electrode layer to the supporting layer of FIG. 1B.

FIG. 3A is a field emission-scanning electron microscopic image of the plane view that varies with the electropolymerization time of the electrode layer on the supporting layer of FIG. 1B.

FIG. 3B is a graph showing changes in the size of the pores with the electropolymerization time of FIG. 3A.

FIG. 4A is a perspective view schematically showing changes in the size of the nanoporous membrane that take place when the nanoporous membrane is oxidized.

FIG. 4B is a perspective view schematically showing changes in the size of the nanoporous membrane that take place when the nanoporous membrane is reduced.

FIG. 5 is a perspective view schematically showing a connection of an impact absorbing layer to the supporting layer of FIG. 1A.

FIG. 6 is a schematic view of a flux measurement system using a nanoporous membrane according to the present invention.

FIG. 7A is a schematic view showing a flux cell with the nanoporous membrane of FIG. 6.

FIG. 7B is a perspective view of the flux cell of FIG. 7A.

FIG. 8A is a graph of the measured flux by the flux measurement system of FIG. 6 upon electrical stimulation when the size of the pores of the nanoporous membrane is 200 nm, and an atomic force microscopic image showing changes in pore size caused by electrical stimulation.

FIG. 8B is a graph of the measured flux by the flux measurement system of FIG. 6 upon electrical stimulation when the size of the pores of the nanoporous membrane is 100 nm, and an atomic force microscopic image showing changes in pore size caused by electrical stimulation.

FIG. 9 is a schematic view of a drug release device including a nanoporous membrane according to the present invention.

FIG. 10 is a graph showing the cumulative concentration of released drug, which varies according to whether the pores are opened or closed and is measured by the drug release device of FIG. 9.

FIG. 11A is a cyclic voltammetry graph showing the process in which the oxidation and reduction of the nanoporous membrane of the present invention are repeated.

FIG. 11B is a field emission-scanning electron microscopic image of the nanoporous membrane after the oxidation and reduction of the nanoporous membrane of FIG. 11A are repeated.

FIG. 12 is a photographed image of the nanoporous membrane after the oxidation and reduction process of FIG. 11A are repeated.

FIG. 13 is a flowchart showing a method for forming a nanoporous membrane according to another exemplary embodiment of the present invention.

### DETAILED DESCRIPTION OF THE EMBODIMENTS

In the following detailed description, only certain exemplary embodiments of the present invention have been shown and described, simply by way of illustration. As those skilled in the art would realize, the described embodiments may be modified in various different ways, all without departing from the spirit or scope of the present invention. Throughout the specification and drawings identical reference numerals refer to identical or similar parts.

In the drawings, the sizes of layers or the like may be exaggerated for clarity. It will also be understood that, when an element is referred to as being “on” another element, it can be directly on the other element, or intervening elements may also be present.

In addition, it will also be understood that, when an element is referred to as being “between” two elements, it can be the only element between the two elements, or other intervening elements may also be present. Further, the same reference numerals are referred to the same elements.

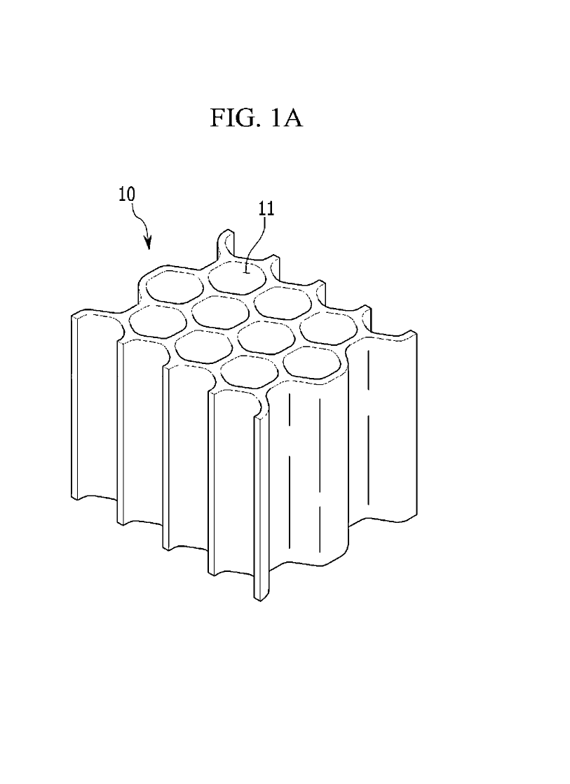

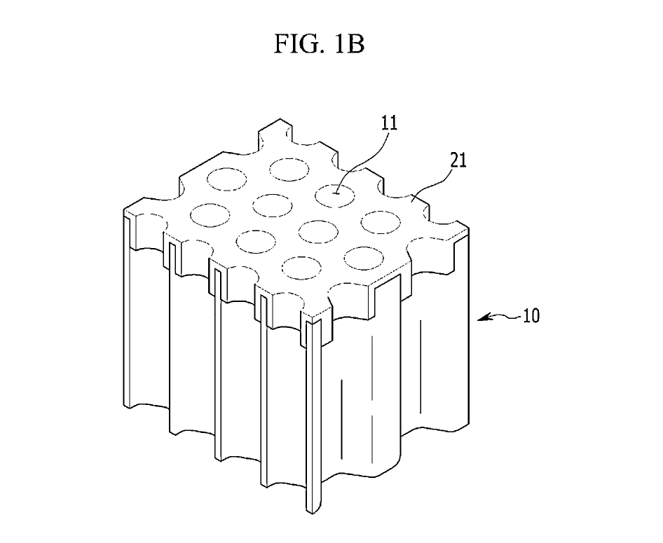

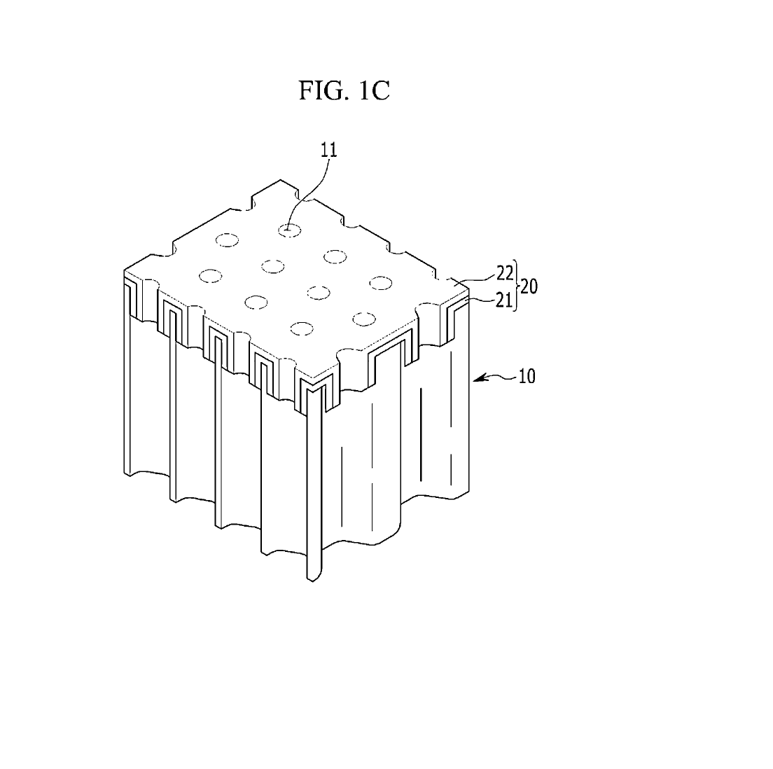

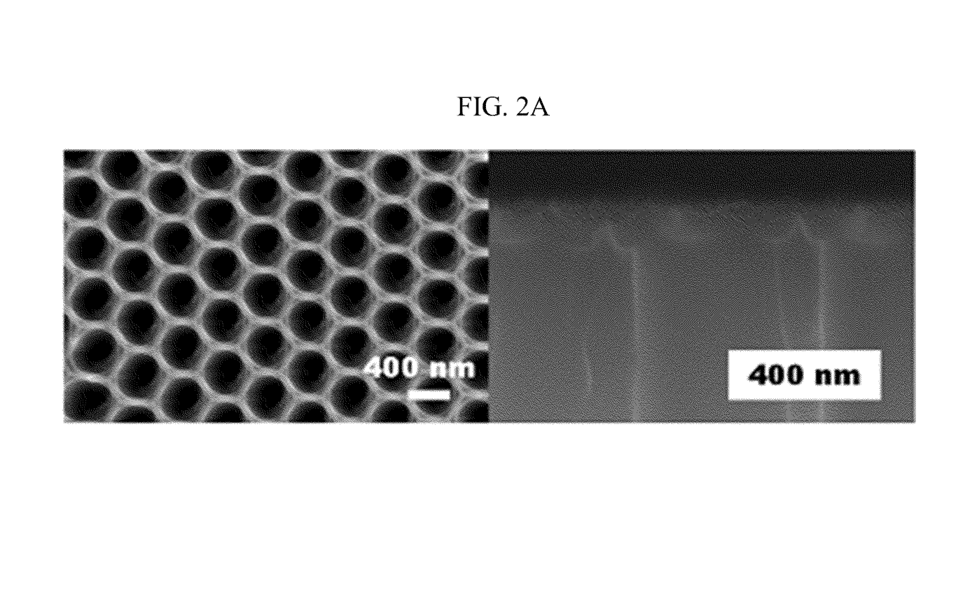

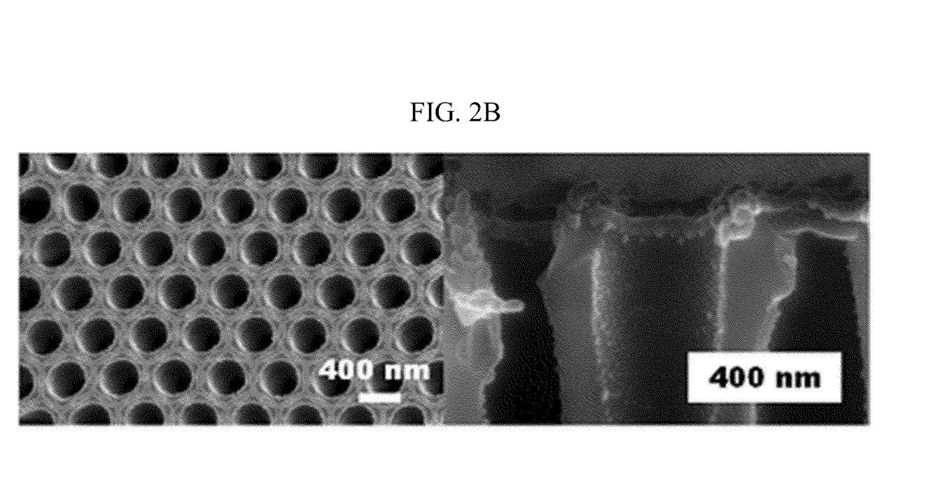

FIG. 1A is a perspective view schematically showing a supporting layer according to the present invention, FIG. 1B is a perspective view schematically showing a connection of an electrode layer to the supporting layer of FIG. 1A, and FIG. 1C is a perspective view schematically showing a connection of a conducting polymer layer to the electrode layer of FIG. 1B. Further, FIG. 2A is a field emission-scanning electron microscopic image of the plane and cross-sectional view of the supporting layer of FIG. 1A, and FIG. 2B is a field emission-scanning electron microscopic image of the plane and cross-sectional view of the connection of the electrode layer to the supporting layer of FIG. 1B.

A nanoporous membrane 100 according to an exemplary embodiment of the present invention will be described in detail with reference to FIG. 1A through FIG. 1C. The nanoporous membrane 100 according to the exemplary embodiment of the present invention includes a supporting layer 10 of a predetermined thickness having a plurality of pores 11 formed therein and an electrically responsive layer 20 connected to around the entrances of the pores 11 formed in the supporting layer 10.

More specifically, the supporting layer 10 according to the exemplary embodiment may be made of anodic aluminum oxide membrane. However, the supporting layer 10 according to the present invention is not limited to those made of anodic aluminum oxide membrane. For example, if pores of a substantially uniform size can be formed, inorganic (metallic and non-metallic) or organic materials can be used to constitute the supporting layer 10.

Moreover, as shown in FIG. 1A, the electrically responsive layer 20 according to the present exemplary embodiment may include an electrode layer 21 and a conducting polymer layer 22 connected to the electrode layer 21.

The electrode layer 21 may be connected to around the entrances of the pores. The electrode layer 21 may be made of conductive material. The conductive material may include gold, but the conductive material is not limited to gold and may include any material through which current can pass.

Moreover, the conducting polymer layer 22 according to the present exemplary embodiment may include a conducting polymer and a dopant.

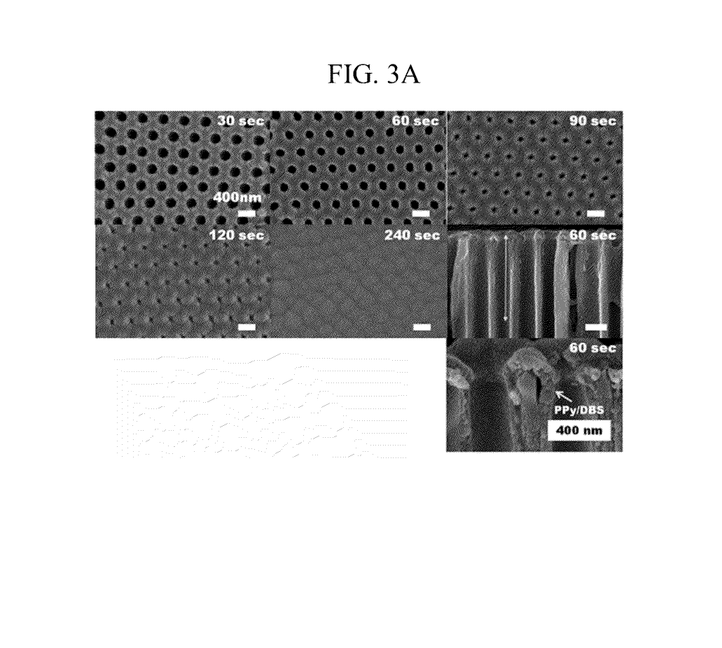

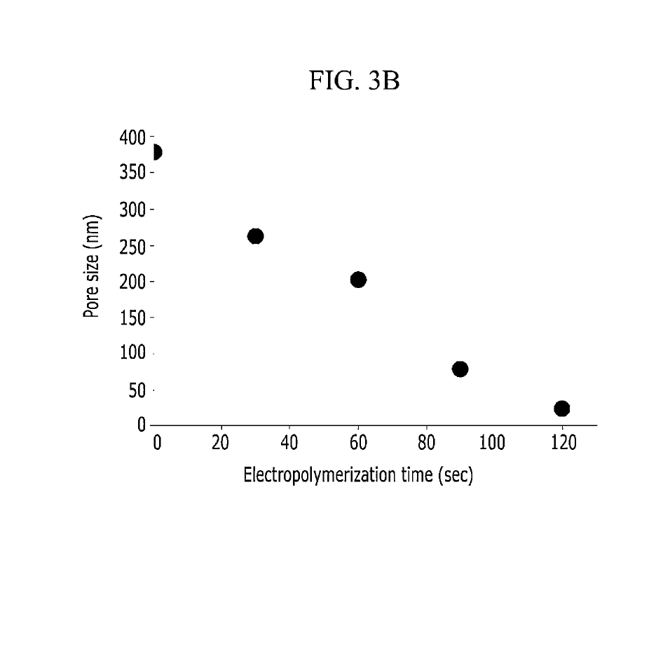

FIG. 3A is a field emission-scanning electron microscopic image of the plane view that varies with the time of polymerization of the electrode layer on the supporting layer of FIG. 1B, and FIG. 3B is a graph showing changes in the size of the pores with the polymerization time of FIG. 3A.

As shown in FIG. 3A and FIG. 3B, the size of the pores 11 may become smaller as the electropolymerization time of the conducting polymer layer 22 is lengthened.

Referring again to FIG. 3A, it can be seen that, when the electropolymerization time of the conducting polymer layer 22 is 60 seconds, the thickness of the conducting polymer layer 22 is approximately 90 nm.

More specifically, the conducting polymer layer 22 according to the present exemplary embodiment may be formed by electrically polymerizing polypyrrole as a conducting polymer and dodecylbenzenesulfonate anions as a dopant (PPy/DBS).

Accordingly, as shown in FIG. 3A, the pores 11 are formed longitudinally in a vertical direction, and the conducting polymer layer 22 (referring to FIG. 3A, P Py/DBS:polypyrrole/dodecylbenzenesulfonate anions refer to the conducting polymer layer) extends 1.5 um from around the entrances of the pores 11 to the entrances of the pores 11. Therefore, the overall flux is high because the length of fluid flow control is small, and the amount of fluid (e.g., drug) passing through the pores 11 may be varied with changes in the size of the pores 11. Moreover, the pores 11 have high density and approximately uniform size, and the conducting polymer layer 22 can quickly respond to electrical stimulation within few seconds.

As a result, it is possible to precisely control the amount of release of liquid such as drug by means of the nanoporous membrane 100 according to the present exemplary embodiment.

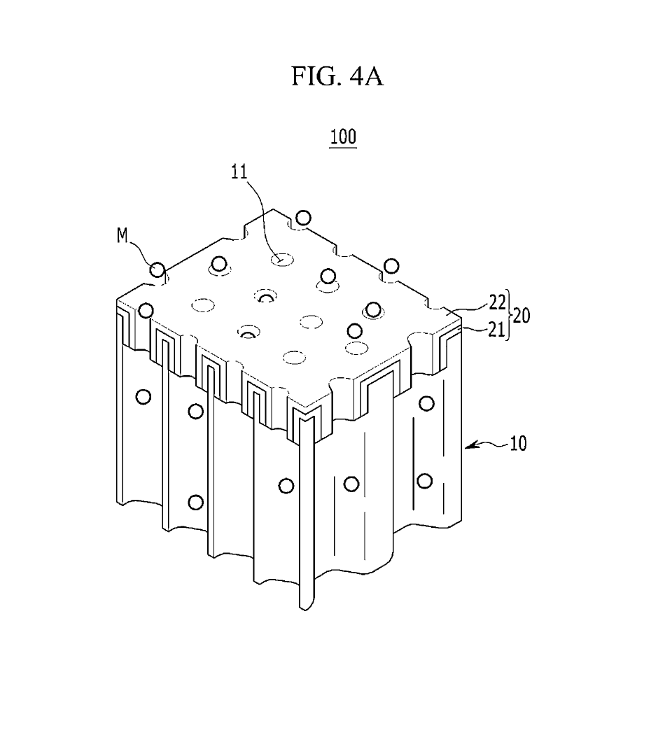

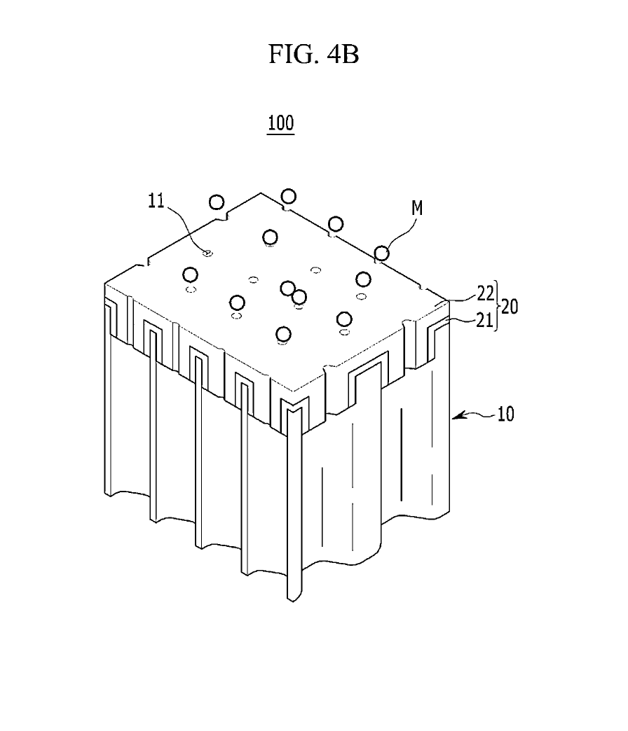

FIG. 4A is a perspective view schematically showing changes in the size of the nanoporous membrane that take place when the nanoporous membrane is oxidized, and FIG. 4B is a perspective view schematically showing changes in the size of the nanoporous membrane that take place when the nanoporous membrane is reduced.

Referring to FIG. 4A and FIG. 4B, the electrically responsive layer 20 may be oxidized or reduced by external electrical stimulation.

More specifically, as shown in FIG. 4A, when the electrically responsive layer 20 is brought into an oxidized state by electrical stimulation, the conducting polymer layer 22 is contracted. Accordingly, the size of the pores 11 becomes larger, thus releasing more liquid (e.g. drug).

Moreover, as shown in FIG. 4B, when the electrically responsive layer 20 is brought into a reduced state by electrical stimulation, the conducting polymer layer 22 is expanded. Accordingly, the size of the pores 11 may become smaller or the pores 11 may be closed, thus leading to a decrease in the amount of release of liquid (e.g., drug) or stopping the liquid release.

Hereinafter, the process of contraction or expansion of the conducting polymer layer 22 depending on the oxidized and reduced state of the electrically responsive layer 20 will be described in more detail.

According to the present exemplary embodiment, polypyrrole and dodecylbenzenesulfonate anions (PPy/DBS) may be polymerized in an oxidized state. Here, polypyrrole has cross-linked chains, and the dodecylbenzenesulfonate anions as the dopant may be larger in size than the space between the cross-linked chains. Accordingly, the dodecylbenzenesulfonate anions may be stabilized in the polypyrrole chains. When the polypyrrole is reduced, hydrated sodium ions (Na⁺) penetrate into the chains of polypyrrole, and therefore the conducting polymer layer 22 is expanded.

As a result, when the conducting polymer layer 22 is brought into the reduced state, the size of the pores 11 can be decreased or the pores 11 can be closed. However, when the conducting polymer layer 22 is brought into the oxidized state, the volume is decreased and the size of the pores 11 returns to the original size, thus making the conducting polymer layer 22 contracted.

Here, hydrated sodium ions (Na⁺) migrate into the cross-linked chains of polypyrrole in order to keep electric neutrality according to the oxidized and reduced state of the polypyrrole chains.

However, if the size of dopant is smaller than the space between the cross-linked chains of polypyrrole, the dopant can be released through the space between the chains when the polypyrrole is in the reduced state, thereby contracting the volume of the conducting polymer layer 22. In this case, if the size of dopant is smaller than the space between the chains of polypyrrole, perchloride ions (ClO₄⁻) can be used as the dopant. As a result, the volume change of the conducting polymer layer 22 according to the oxidized or reduced state varies with the type of the dopant ion to be electrically polymerized. Also, the volume change of the conducting polymer may vary with the solution, type of ions, and pH used for electrical stimulation.

The oxidation and reduction reactions of the electrically responsive layer 20 may occur reversibly by varying the applied electricity. Therefore, the amount of release of liquid (e.g., drug) can be controlled relatively freely.

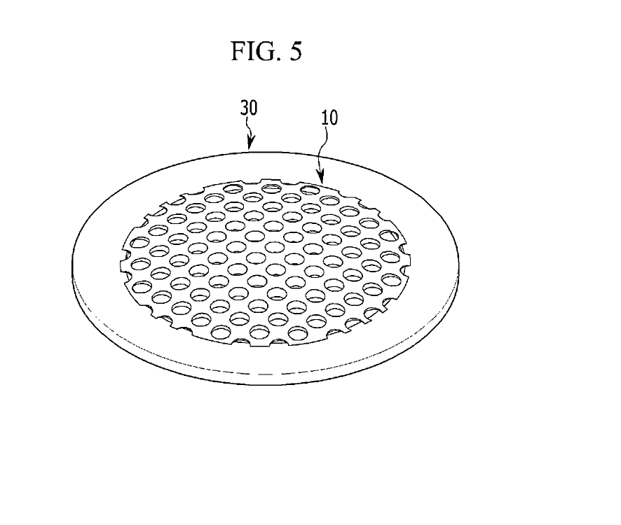

FIG. 5 is a perspective view schematically showing a connection of an impact absorbing layer to the supporting layer of FIG. 1A.

Referring to FIG. 5, the nanoporous membrane 100 according to the present exemplary embodiment may further include an impact absorbing layer 30 connected to the supporting layer 10. Accordingly, external impact can be absorbed by the impact absorbing layer 30 connected to around the supporting layer 10 which is fragile. Here, the impact absorbing layer 30 may include polymer. As a result, the supporting layer 10 to which the impact absorbing layer 30 is connected may be stably installed in a device for use.

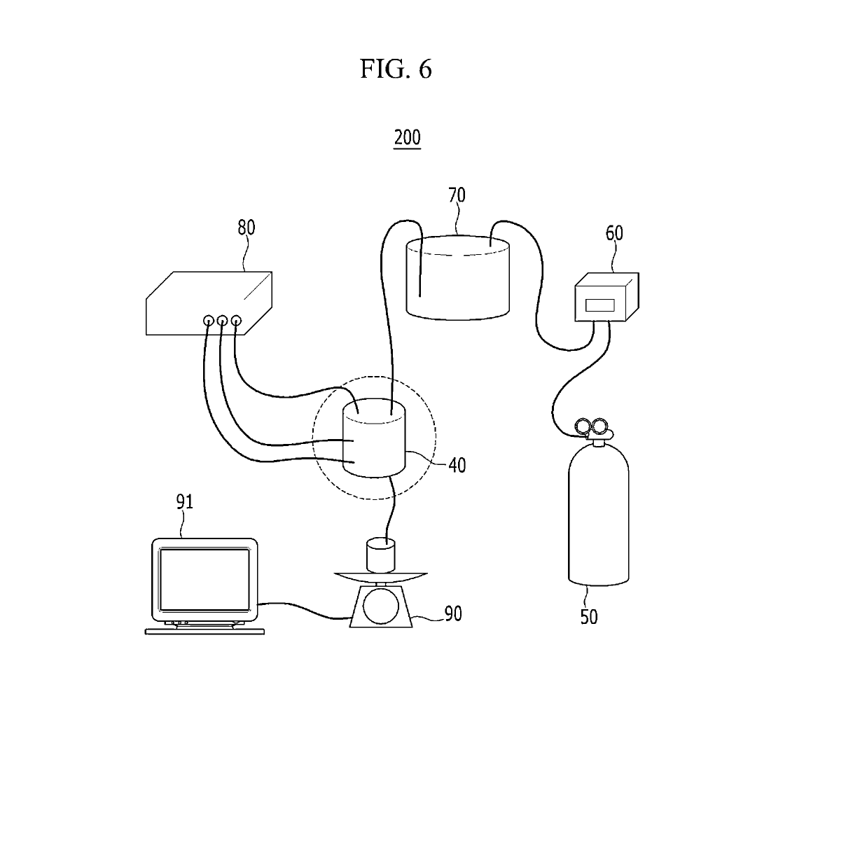

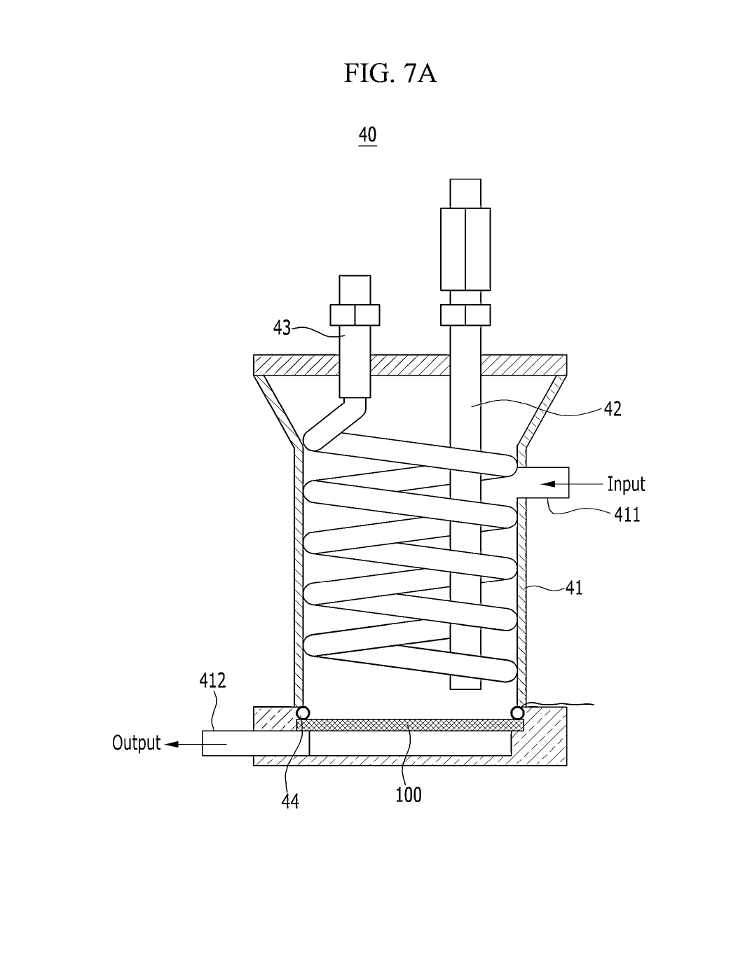

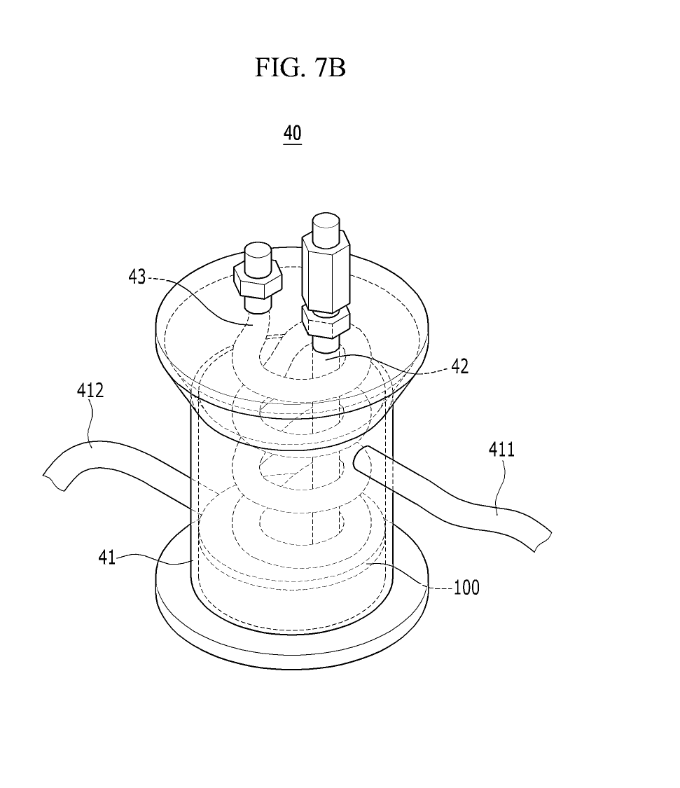

FIG. 6 is a schematic view of a flux measurement system using a nanoporous membrane according to the present invention, FIG. 7A is a schematic view showing a flux cell with the nanoporous membrane of FIG. 6, and FIG. 7B is a perspective view of the flux cell of FIG. 7A.

Referring to FIG. 6, the flux measurement system according to the present exemplary embodiment includes a flux cell 40, a nitrogen (N₂) reservoir 50, a barometer 60, a fluid reservoir 70, a potentiostat 80, a balancing device 90, and a control device 91.

Referring to FIG. 6, the flux measurement system according to the present exemplary embodiment will be described. An internal pressure of the flux cell 40 is controlled by N₂ of the nitrogen (N₂) reservoir 50. The pressure of the nitrogen (N₂) can be controlled by the barometer 60. The nitrogen (N₂) discharged from the nitrogen (N₂) reservoir 50 may be used to control the pressure of the flux cell 40 by means of the fluid reservoir 70. Hence, the flux cell 40 and the fluid reservoir 70 have the same pressure.

Here, the internal pressure of the flux cell 40 may be kept to be about 0.1 bar higher than the air pressure. As a result, the fluid inside the flux cell 40 can be flow to the outside due to the difference between the internal pressure of the flux cell 40 and the air pressure. Moreover, the fluid reservoir 70 can play the role of supplying fluid to the flux cell 40.

Further, the potentiostat 80 may switch the nanoporous membrane to the oxidized or reduced state by applying electrical stimulation (e.g., −0.1 V or 1.1 V) to the flux cell 40.

In addition, when the nanoporous membrane is in the oxidized or reduced state, the balancing device 90 can measure the mass of the fluid flowing through the flux cell 40. The measured mass of the fluid can be converted into a volume by means of its density.

Besides, the control device 91 can automatically record the mass of the fluid discharged from the flux cell 40 depending on a change in the oxidized or reduced state.

Also, referring to FIG. 7A and FIG. 7B, the flux cell 40 according to the present exemplary embodiment may include a case 41 including a fluid inlet 411 and a fluid outlet 412, a reference electrode 42, a counter electrode 43, and a nanoporous membrane 100 which used as a working electrode.

More specifically, the nanoporous membrane 100 can be used as the working electrode, platinum (Pt) can be used as the counter electrode 43, and Ag/AgCl or Ag wire can be used as the reference electrode. Moreover, the inside of the case 41 can be filled with an aqueous solution containing sodium ions.

When the fluid of the aqueous solution containing sodium ions is supplied at a same speed with outlet rate into the fluid inlet 411 of the flux cell, the fluid passing through the nanoporous membrane 100 can be discharged through the fluid outlet 412. In this case, the nanoporous membrane 100 is contracted in the oxidized state (e.g., −0.1 V), and expanded in the reduced state (e.g., 1.1 V).

However, according to the present exemplary embodiment, oxidation is not limited to always occurring at a negative voltage, while reduction is not limited to always occurring at a positive voltage.

Accordingly, when the nanoporous membrane 100 is contracted by oxidation, the pores 11 become larger in size and the discharge amount of the fluid increases; whereas when the nanoporous membrane is expanded by reduction, the pores 11 become smaller in size and the discharge amount of the fluid decreases.

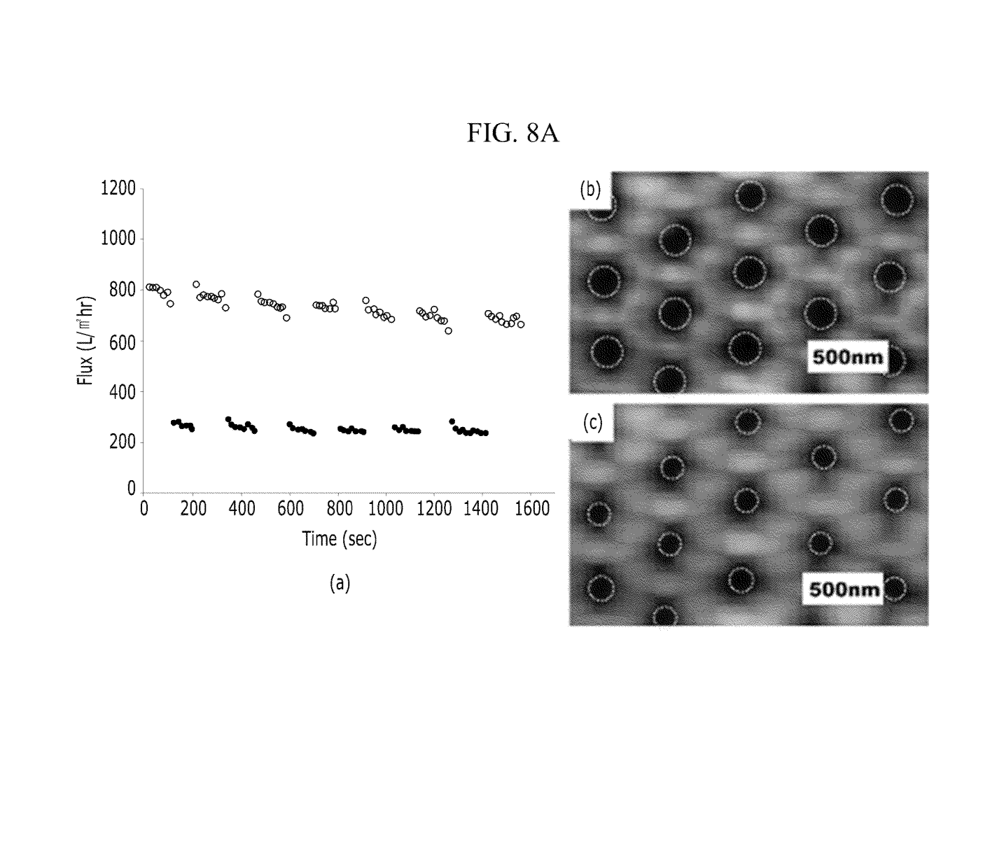

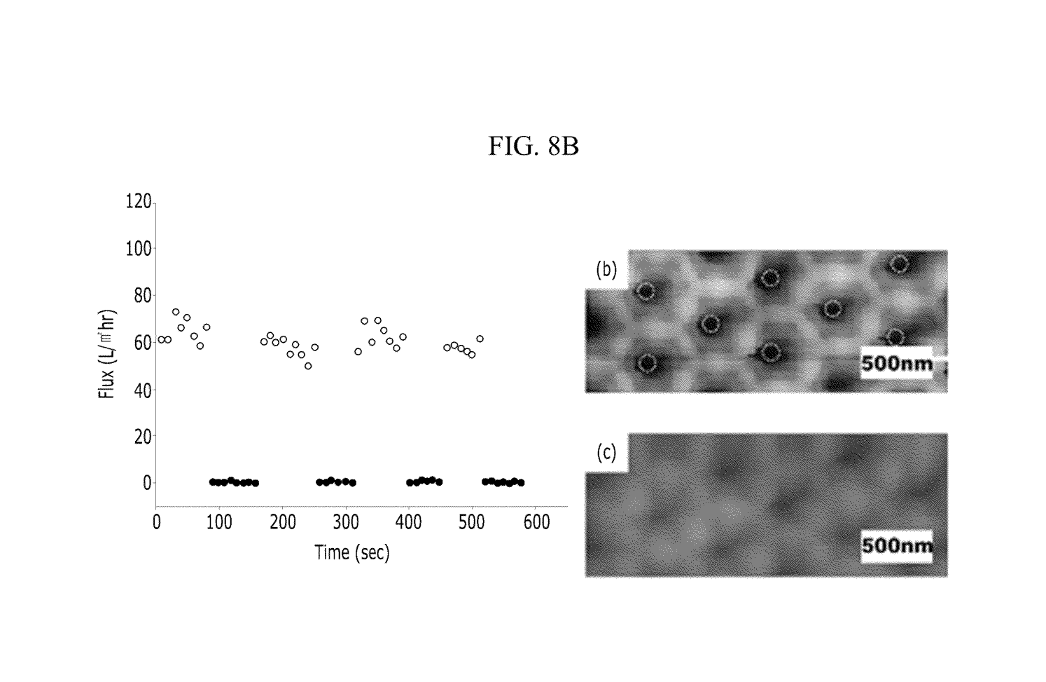

FIG. 8A is a graph of the measured flux by the flux measurement system of FIG. 6 upon electrical stimulation when the size of the pores of the nanoporous membrane can be switched to 200 nm and 100 nm in response to electrical stimulation, and an atomic force microscopic image showing changes in pore size under each condition.

More specifically, referring to FIG. 8A, part b of FIG. 8A shows the measurement when the size of the pores 11 is 200 nm because of the oxidation and contraction of the conducting polymer layer 22. Referring to higher region of part a of FIG. 8A, it can be observed that the flux discharged through the pores 11 of part a is approximately 730 (L/m²h).

Moreover, part c of FIG. 8A shows the measurement when the size of the pores 11 becomes smaller in size because of the reduction and expansion of the conducting polymer layer 22. Referring to lower region of part a of FIG. 8A, it can be seen that the flux is approximately 250 (L/m²h).

As a result, it is found out that, when the size of the pores 11 is changed by oxidation and reduction, the flux is also changed to a large degree according to the change in the pore size.

Referring to FIG. 8B, it can be observed that, as the size of the pores 11 is changed from 100 nm (see part b of FIG. 8B) to approximately 0 nm (see part c of FIG. 8B), the flux is changed from approximately 60 (L/m²h) to 0 (L/m²h) (see part a of FIG. 8B).

As a result, by means of the nanoporous membrane 100 according to the present exemplary embodiment, the flux can be controlled by controlling the size of the pores 11.

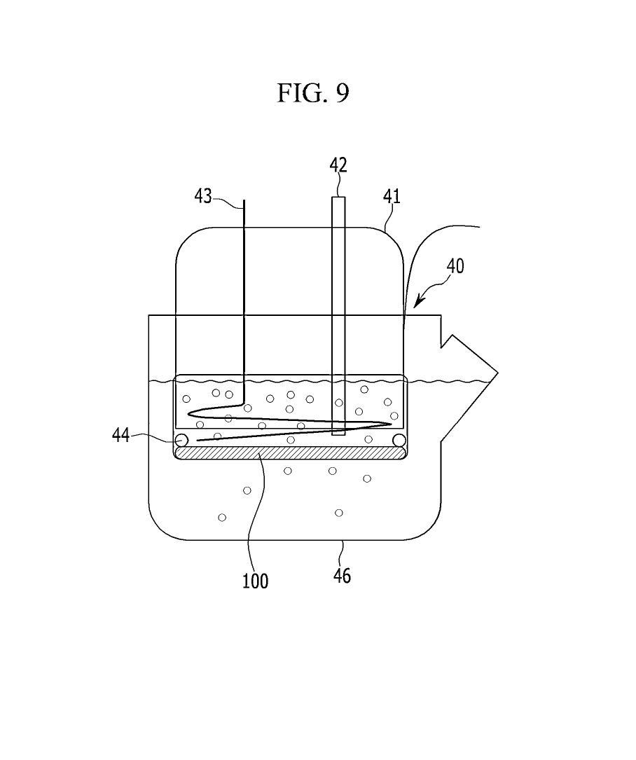

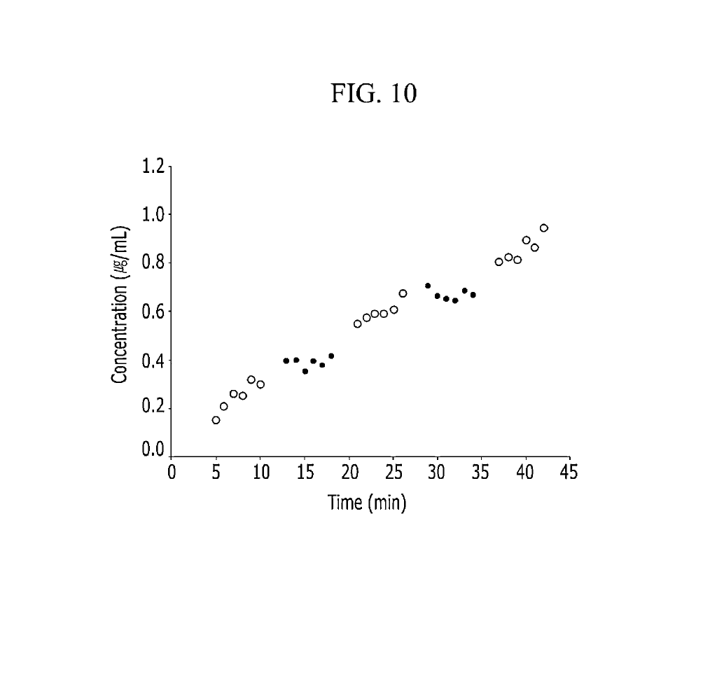

FIG. 9 is a schematic view of a drug release device including a nanoporous membrane according to the present invention, and FIG. 10 is a graph showing the cumulative concentration of released drug, which varies according to whether the pores are opened or closed and is measured by the drug release device of FIG. 9.

Referring to FIG. 9, the drug release device according to the present exemplary embodiment is the same as the flux cell described in FIG. 7A except existence of pressure. In this exemplary embodiment, the flux cell will be described as the drug release device for convenience of explanation. The flux cell and the drug release device have the same configuration, and description of the same configuration will be omitted.

As shown in FIG. 9, the drug release device 40 is held in a basket 46 filled with sodium ions. At this point, if the drug release device 40 repeats oxidation or reduction, the model drug (e.g., FITC-BSA (Fluorescein IsoThioCyanate-labeled Bovine Serum Albumin) inside the drug release device may pass through the pores 11 and be discharged toward the basket. Accordingly, the amount of the drug discharged toward the basket may vary according to the opening and closing degree of the pores 11. Here, the drug inside the drug release device may be discharged from the inside of the drug release device 40 to the basket 46 because of the diffusion of the drug caused by a difference in drug concentration between the drug release device 40 and the basket 46.

Referring to FIG. 10, the period of time between around 5 to 10 minutes shows a change in drug concentration when the pores are opened, and the period of time between around 15 to 20 minutes shows a change in drug concentration when the pores are closed. From this, it can be found out that, while the drug concentration gradually increases when the pores are opened, there is no change in drug concentration when the pores are closed.

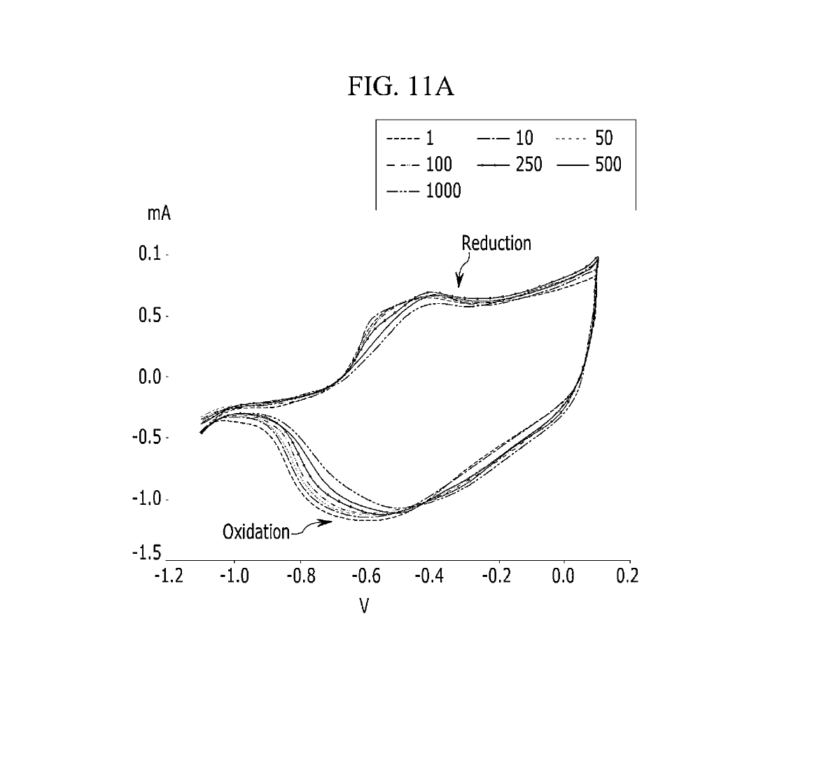

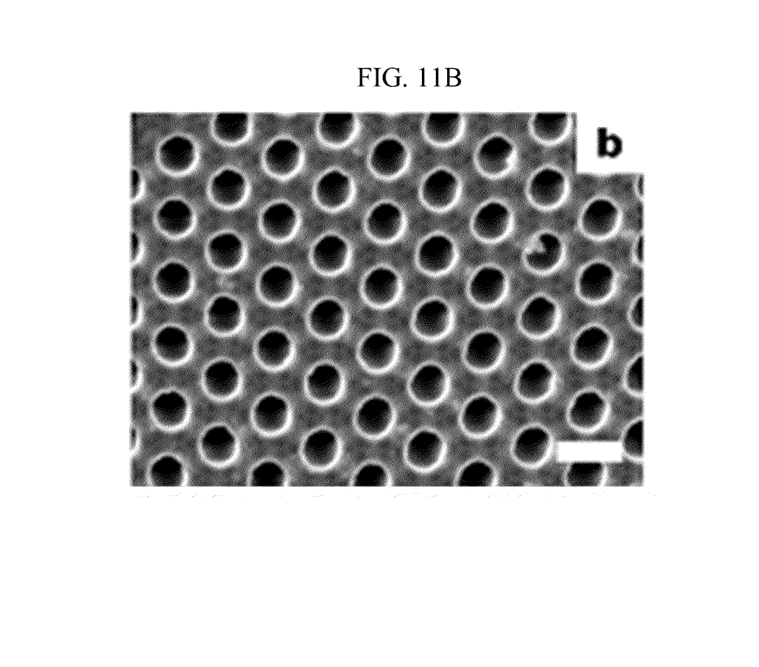

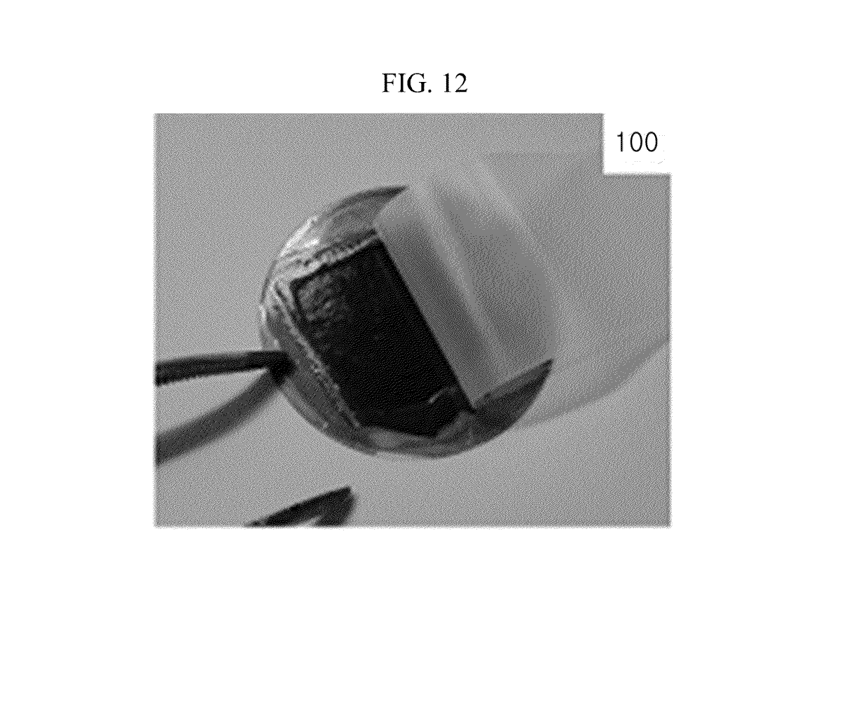

FIG. 11A is a cyclic voltammetry graph showing the process in which the oxidation and reduction of the nanoporous membrane of the present invention are repeated, FIG. 11B is a field emission-scanning electron microscopic image of the nanoporous membrane after the oxidation and reduction of the nanoporous membrane of FIG. 11A are repeated, and FIG. 12 is a photographed image of the nanoporous membrane after the oxidation and reduction of FIG. 11A are repeated.

Referring to FIG. 11A, it can be seen that the nanoporous membrane 100 according to the present exemplary embodiment remains chemically stable even it undergoes the oxidation or reduction process about more than 1,000 times.

Moreover, referring to FIG. 11B, it can be seen that the physical state of the nanoporous membrane 100 is constant without a significant change from the early stage.

Further, referring to FIG. 12, a peel-off test using 3M scotch tape confirmed that the nanoporous membrane 100 is mechanically stable and maintained.

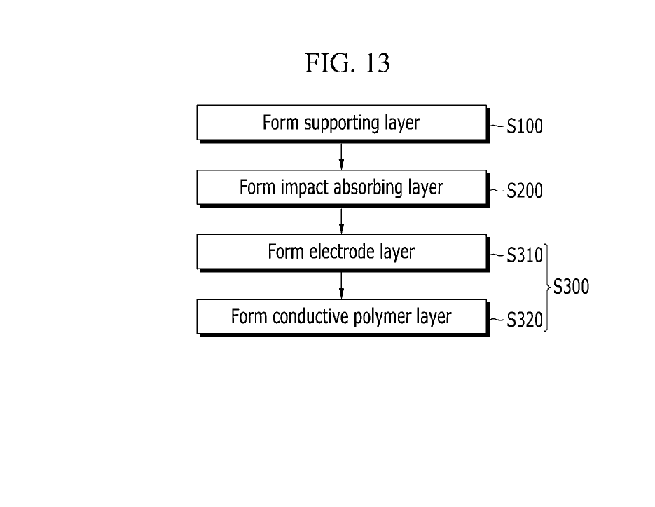

FIG. 13 is a flowchart showing a method for forming a nanoporous membrane according to another exemplary embodiment of the present invention.

Referring to FIG. 13, the method for forming the nanoporous membrane 100 according to the present exemplary embodiment includes the step S100 of forming a supporting layer 10 with a plurality of pores 11, the step S200 of forming an impact absorbing layer 30, and the step S300 of forming an electrically responsive layer 20.

More specifically, the step S100 of forming a supporting layer 10 according to the present exemplary embodiment may include the step of forming pores using an anodic aluminum oxide membrane.

Anodic aluminum oxide can control the inter-pore distance and the pore size depending on the type of electrolyte used for oxidation and an applied voltage, and also can control the length of the pores according to oxidation time.

Also, because arranged pores are blocked by aluminum after direct anodic oxidation of an aluminum plate, a supporting layer having uniform pores can be obtained through aluminum removal using a copper chloride solution, barrier layer removal using phosphoric acid, and pore widening process.

Therefore, the nanoporous membrane 100 according to the present exemplary embodiment can include a supporting layer 10 having an inter-pore distance of 500 nm, a pore size of 410 nm, and a pore length of 60 um by anodically oxidizing aluminum at 195 V under a 0° C. phosphoric acid solution.

As a result, as shown in FIG. 2A, a plurality of pores can be formed in a substantially hexagonal array on an anodic aluminum oxide membrane.

Here, the anodic aluminum oxide membrane can be formed arranged pores array by self-assembly.

Moreover, the method for forming the nanoporous membrane according to the present exemplary embodiment may further include the step S200 of forming an impact absorbing layer on the supporting layer 10.

Hence, the supporting layer 10 to which the impact absorbing layer 30 is connected can be stably installed in a device for use.

Moreover, the step S300 of forming an electrically responsive layer 20 according to the present exemplary embodiment may include the step S310 of forming an electrode layer connected to around the entrances of the pores 11 and the step S320 of forming a conducting polymer layer 22 connected to the electrode layer 21.

The electrode layer 21 may be made of conductive material. The conductive material may include gold.

Gold may be deposited around the entrances of the pores 11 by either thermal deposition or sputtering. In this case, the deposition speed of the electrode layer 21 may be 3 to 5 A/sec, and the deposition thickness may be 40 nm.

Accordingly, as shown in FIG. 2B, the size of the pores after the deposition of the electrode layer 21 around the pores of the supporting layer 10 may become smaller by approximately 30 nm.

Further, the conducting polymer layer 22 according to the present exemplary embodiment may include a conducting polymer and a dopant.

More specifically, the conducting polymer may include polypyrrole, and the dopant may include dodecylbenzenesulfonate anions. That is, the conducting polymer layer 22 may be formed by electrically polymerizing polypyrrole (PPy/DBS:polypyrrole/dodecylbenzenesulfonate anions) including dodecylbenzenesulfonate anions.

For an electropolymerization method for forming the conducting polymer layer 22 according to the present exemplary embodiment, a three-electrode system can be used. That is, by immersing the electrode layer 21 deposited supporting layer 10, to which a reference electrode (Ag/AgCl), a counter electrode (Pt), and a working electrode (electrode layer 21) are connected, in an aqueous solution containing 0.25M pyrrole monomer and 0.1 M NaDBS solved therein, and applying a 0.6V voltage, the conducting polymer layer 22 can be uniformly formed over the entire areas of the electrode layer 21 along the profile the electrode layer 21.

The thickness of the conducting polymer layer 22 may increase in linear proportion with voltage application time. Accordingly, it is also possible to control the size of the pores formed in the nanoporous membrane 100 by controlling electropolymerization time.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

<Description of symbols>

| | |
|---|---|
| nanoporous membrane: 100 | supporting layer: 10 |
| pore: 11 | electrically responsive layer: 20 |
| electrode layer: 21 | conducting polymer layer: 22 |
| impact absorbing layer: 30 | drug release device: 40 |
| nitrogen reservoir: 50 | barometer: 60 |
| Fluid reservoir: 70 | potentiostat: 80 |
| balancing device: 90 | control device: 91 |

1. A nanoporous membrane comprising:
a supporting layer with a plurality of pores; and
an electrically responsive layer that is connected to around the entrances of the pores and undergoes a volume change by oxidation or reduction caused by electrical stimulation to thereby lead to a change in pore size a size of the entrances of the pores.

2. The nanoporous membrane of claim 1, wherein the supporting layer is made of anodic aluminum oxide membrane, and
the electrically responsive layer comprises an electrode layer connected to around the entrances of the pores and a conducting polymer layer that is connected to the electrode layer and undergoes a volume change by oxidation or reduction due to electricity applied to the electrode layer.

3. The nanoporous membrane of claim 2, wherein the electrode layer comprises gold, and gold is formed around the entrances of the pores by either thermal deposition or sputtering.

4. The nanoporous membrane of claim 2, wherein the conducting polymer layer comprises a conducting polymer and a dopant.

5. The nanoporous membrane of claim 4, wherein the conducting polymer comprises polypyrrole, and the dopant comprises dodecylbenzenesulfonate anions.

6. The nanoporous membrane of claim 1, further comprising a impact absorbing layer connected to the supporting layer.

7. The nanoporous membrane of claim 6, wherein the impact absorbing layer comprises polymer.

8. The nanoporous membrane of claim 1, wherein the electrically responsive layer decreases in volume if oxidized by electrical stimulation.

9. The nanoporous membrane of claim 1, wherein the electrically responsive layer increases in volume if reduced by electrical stimulation.

10. A method for forming a nanoporous membrane, the method comprising:
forming a supporting layer with a plurality of pores; and
forming an electrically responsive layer that is connected to around the entrances of the pores and oxidized or reduced by electrical stimulation.

11. The method of claim 10, wherein the forming of a supporting layer comprises forming pores using an anodic aluminum oxide membrane.

12. The method of claim 10, wherein the forming of an electrically responsive layer comprises:
forming an electrode layer connected to around the entrances of the pores; and
forming a conducting polymer layer connected to the electrode layer.

13. The method of claim 12, wherein the electrode layer comprises gold, and gold is formed around the entrances of the pores by either thermal deposition or sputtering.

14. The method of claim 12, wherein the forming of an electrically responsive layer comprises electrically polymerizing the oxidized conducting polymer with the dopant.

15. The method of claim 12, wherein the conducting polymer comprises polypyrrole, and the dopant comprises dodecylbenzenesulfonate anions.

16. The method of claim 10, further comprising connecting the impact absorbing layer to the supporting layer.

17. The nanoporous membrane of claim 7, wherein the electrically responsive layer decreases in volume if oxidized by electrical stimulation.

18. The nanoporous membrane of claim 7, wherein the electrically responsive layer increases in volume if reduced by electrical stimulation.
